(19) United States
(12) Plant Patent Application Publication
(10) Pub. No.: US 2003/0079262 P1
(43) Pub. Date: Apr. 24, 2003

(54) Peach tree named 'Plapiomel'

(76) Inventor: Jose Miguel Arias Lopez, Tudela (ES)

Correspondence Address: CHRISTIE, PARKER & HALE, LLP., P.O. BOX 7068, PASADENA, CA, 91109-7068, US

(21) Appl. No.: 10/185,662

(22) Filed: Jun. 27, 2002

(30) Foreign Application Priority Data
Oct. 23, 2001 (EP) 2001/1661

(51) Int. Cl. A01H 5/00

(52) U.S. Cl. PLT/197

(57) ABSTRACT

A new and distinct variety of Peach tree characterized by its low chilling requirement, early to mid-season ripening, having attractive clingstone fruit having yellow fleshed, medium-high firm, good quality fruit.

### BACKGROUND OF THE INVENTION

The new variety of Peach tree was created in a breeding program by crossing two parents; in particular, by crossing as seed parent a variety designated ‘89-062’ (unpatented) and as pollen parent a variety designated ‘91-002’ (unpatented). Both, female and male, are components of a parent collection from a selection made between plants issued from seeds got in a free pollination in a population of different origin done in 1989 and 1991. Both parental varieties are property and have not been commercialized.

The seeds resulting from this controlled hybridization were germinated in a greenhouse in the spring of 1995 and planted in a field on the farm of La Mogalla in Cartaya (Huelva), Spain, 7° W., 37° N., 45 feet elevation. The seedlings fruited during the Spring of 1997, one designated ‘95.03.003-P,’ (unpatented) was selected for its low chilling requirement (250 to 300 hours) early to mid-season ripening, attractive fruit shape and color, yellow flesh, medium-high firm fruit, and good fruit quality. During 1998, the original plant selection was propagated asexually, at the above noted location, by budding onto standard Peach rootstock variety designated ‘GF-677’ (non patented) and a test plot of 4 plants was established.

The new variety has been asexually multiplied several times since 1999 at this location by budding onto the standard Peach rootstock variety designated as ‘GF-677’ (unpatented) and no incompatibility with Peach rootstock has occurred following budding. During all asexual reproduction, the characteristics of the original plant have been maintained and no aberrant phenotypes have appeared.

### SUMMARY OF THE INVENTION

The new Peach tree variety differs from its parents and other known cultivars of Peach Trees by producing clingstone fruits with a low chilling requirement (250 to 300 hours) and an early to mid-season ripening date. The fruits of the new variety are ripe for commercial harvesting and shipment between approximately May 30^{th} to May 15^{th}. These harvesting dates are approximately 25 days earlier than the harvest dates of the commercial Peach variety ‘VISTA’ (U.S. Plant Pat. No. 9,549), approximately 13 days earlier than the harvest dates of the commercial Peach variety ‘REDHAVEN’ (unpatented) , approximately 15 days earlier than the harvest dates of the commercial peach variety ‘RICH LADY’ (U.S. Plant Pat. No. 7,290), and approximately 20 days later than the harvest dates of the commercial peach variety ‘RICH MAY’ (U.S. Plant Pat. No. 7,432).

### COMPARISON WITH OTHER VARIETIES

‘PLAPIOMEL’ differs from Peach trees of ‘VISTA,’ ‘RICH MAY’ and ‘RICH LADY’ by its lower chilling requirement (200 to 300 hours); whereas ‘VISTA’ has a 700 to 800 hour chilling requirement; ‘RICH MAY’ has a 800 to 900 hour chilling requirement; and ‘RICH LADY’ has a 750 to 850 hour chilling requirement.

‘PLAPIOMEL’ produces round shaped clingstone fruit with a slightly apical tip; whereas ‘VISTA’ produces semi-freestone fruit with an ovoid shape; ‘RICH MAY’ produces more firm, ovoid shaped fruit with a strong apical tip. The fruit of ‘RICH LADY’ also have an ovoid shape.

The surface of the fruit of the new variety is covered approximately 60% to 80% with a red over a yellow-greenish color; whereas 100% of the fruit surface of ‘RICH LADY’ shows a red color.

### BRIEF DESCRIPTION OF THE DRAWINGS

The accompanying photographic illustrations show typical specimens of the new variety as true to color as is reasonably possible to make in illustrations of this character.

FIG. 1 shows typical flowers of the new variety.

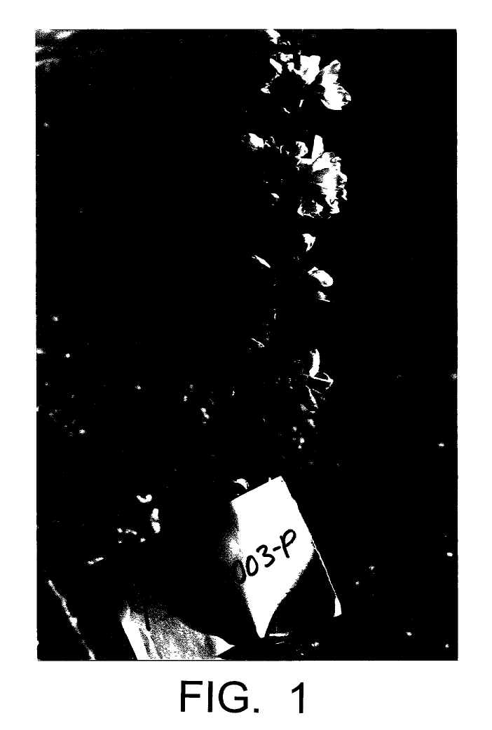

FIG. 2 shows the coloration of the dorsal and ventral leaves of the new variety.

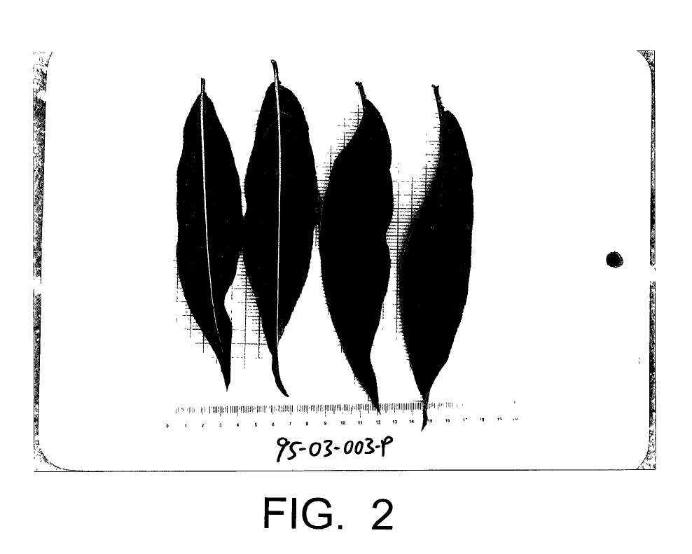

FIG. 3 shows the external coloration of fruits of the new variety when sufficiently matured for harvesting and shipment and one fruit of the new variety dissected in the equatorial plane to illustrate the flesh and stone characteristics.

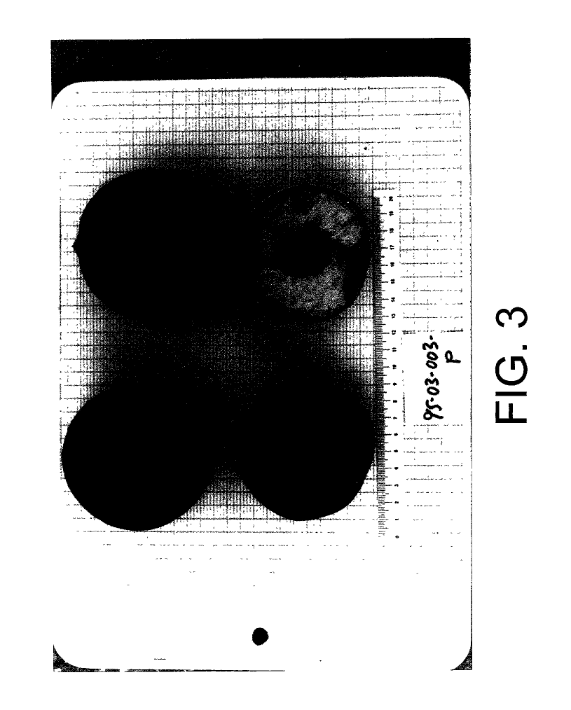

### DESCRIPTION OF THE NEW VARIETY

The following observations and descriptions are of plants grown at the orchard previously described, located near the town of Cartaya, Huelva, Spain. Observations were made of the original seedling tree on its own root during the growing years of 1997 to 2001.

In this description, it is to be understood that references of the dimensions, sizes, colors, etc. of the botanical and phonological characteristics of the new variety are approximations of averages set forth as accurately as practical. Phenotypic expression may vary with light intensity and cultural and environmental conditions. The color references used in this description, are to The Royal Horticultural Society Colour Chart (RHSCC) and terminology used in the color descriptions herein refers to plate numbers in said color chart.

Tree

Size.—Generally average to above-average as compared to other common Peach cultivars.

Productivity.—Approximately 27000 to 29000 Kgrs/Ha.

Form.—Considered upright to upright, spreading in form.

Height.—Original seedling tree reached approximately 3.2 m at end of 2001 growing season.

Width.—Original seedling reached approximately 1.80 m at end of 2001 growing season.

Current season growth.—Approximately 0.95 to 1.1 m.

Regularity of bearing.—Regular.

Trunk

Thickness.—Approximately 110 mm Diameter: Approximately 15 cm when measured at a distance above the soil level at end of 2001 growing season.

Bark texture.—Considered moderately rough with numerous scarf skin flat oval lenticels present Color: Grey Group near 201C to 201B.

Branches

Size.—Considered medium for the species.

Diameter.—About 65.4 mm when measured during the 3^{rd} year after grafting.

Surface texture.—Average Current Season Shoots: Substantially glabrous Color: Greyed-green Group near 194B to 195B.

Mature branches.—Appearing furrowed on wood which is several years old Color: Greyed-green Group near 197C to 197B.

Leaf

Size.—Considered average for the species; leaf measurements taken from vigorous upright current season growth at approximately mid-shoot Length: Approximately 140 to 160 mm Width: Approximately 38 to 46 mm Thickness: Approximately 1 to 2 mm.

Base shape.—Slightly oblique.

Form.—Lanceolate.

Tip form.—Acuminate.

Color.—Upper Side: Green Group near 146A to 147A Underside: Yellow-Green Group near 146D to 146B.

Texture.—Glabrous.

Margins.—Crenate, generally uniform.

Leaf petiole.—Considered medium-long Length: Approximately 10 to 14 mm Diameter: Approximately 1.5 to 2 mm.

Leaf glands.—Reinform; generally 2 to 4 per side Length: Approximately 1.0 to 1.5 mm Width: Approximately 0.5 to 1.0 mm.

Inflorescence

Flowers.—Bloom occurs prior to vegetative bud break; generally double individual flowers at a single node; perfect self-fertile.

Blooming time.—Considered medium in relation to other Peach cultivars.

Date of bloom.—First, February 9^{st}; Full, February 16^{th}.

Type.—Roseate.

Flower diameter.—Approximately 41 to 45 mm at full bloom.

Bloom quantity.—Considered very abundant.

Petalage.—Considered large for the species Length: Approximately 17 to 19 mm Width: Approximately 17 to 19 mm Shape: Rounded.

Petal count.—Nearly always 5.

Texture.—Glabrous.

Color.—Red-Purple Group near 69D to 69C.

Apex.—The petal apices appear domed.

Stamens.—Numerous; with pollen present; fertile and abundant.

Fruit: In firm ripe condition at full commercial maturity, first fruit picked on approximately May 30^{th}; last pick of same fruit in 2001 was approximately June 15^{th} in Cartaya, Huelva, Spain conditions

Size.—Generally considered large in size, slightly not uniform Cheek Diameter: Approximately 70 to 88 mm Suture Diameter: Approximately 72 to 90 mm Axial diameter: Average about 76 to 78 mm.

Form.—Rounded, slightly flat; generally slightly not-uniform.

Suture.—Extending from base to apex, suture appears as a very thin line at same level as skin.

Stem cavity size.—Considered small for the species Width: Approximately 21 to 22 mm Length: Approximately 28 to 30 mm Depth: Approximately 16 to 18 mm.

Form.—Ovoid.

Fruit base.—Slightly concave and uniform.

Fruit apex.—Rounded with a slight apical tip.

Fruit skin.—Average in thickness Surface Texture: Very little pubescence Skin Acidity: Neutral.

Tenacious to flesh.—Yes; at commercial maturity.

Tendency to crack.—Not observed Skin Color: Approximately 60% to 80% of fruit surface Red Group near 45B to 46A; not brilliant and about 20% to 40% of surface Yellow-green Group near 145C to 145D.

Firmness.—3.5 to 4.5 Kg/cm²= Resistance to penetration measured in Kilograms (Kg/cm²) obtained by Penetrometer ROZE Mod. Arbelette, with a 50 mm² section head.

Flesh color.—Yellow Group near 11B to 11A.

Flesh fibers.—Not present throughout flesh at maturity.

Flesh texture.—Generally melting.

Flavor.—Considered sweet; medium acidic; soluble solids, as °Brix, about 11 to 12.

Aroma.—Pleasant and medium.

Eating quality.—Very good to excellent; well above average when compared to other common commercial varieties.

Stone.—Attachment: Clingstone at full commercial maturity Stone Size: Large for the species Length: Approximately 35 to 37 mm Width: Approximately 25 to 27 mm Thickness: Approximately 19 to 20 mm Stone Form: Ovoid-elongated Stone Color: Yellow-Orange Group near 22A to 26B Tendency to Split: None observed Kernel: Form is ovoid-elongated. Length: Approximately 15 to 16 mm; Width: Approximately 10 to 11 mm.

Use: Early to mid-season maturity producing a highly attractive colored, medium-high firm fruit

Keeping Quality: Fruit stored well up to 15 to 20 days after harvest at temperatures of about 1° C.

Resistance to Insects and Disease: No particular susceptibilities were noted

Shipping Quality: Well above average

The new variety of Peach tree possesses the characteristics described above, as a result of the growing conditions prevailing in Cartaya, Huelva, Spain. It is to be expected that variations in these characteristics may occur when farmed in areas with different climatic conditions, different soil types, and/or varying cultural practices.

I claim:

1. A new and distinct variety of Peach tree substantially as illustrated and described herein.
